(19) United States
(12) Patent Application Publication
(10) Pub. No.: US 2020/0197270 A1
(43) Pub. Date: Jun. 25, 2020

(54) ORAL CARE COMPOSITIONS

(71) Applicant: Colgate-Palmolive Company, New York, NY (US)

(72) Inventors: Saide Tang, Princeton, NJ (US); Lin Fei, Kendall Park, NJ (US); Suman Chopra, Monroe, NJ (US); Neelima Utgikar, Dombivli (IN)

(73) Assignee: Colgate-Palmolive Company, New York, NY (US)

(21) Appl. No.: 16/721,900

(22) Filed: Dec. 19, 2019

Related U.S. Application Data
(60) Provisional application No. 62/782,702, filed on Dec. 20, 2018.

(51) Int. Cl. A61K 8/25; A61Q 11/00; A61K 8/02; A61K 8/27

(52) U.S. Cl. CPC A61K 8/25; A61Q 11/00; A61K 8/0241; A61K 8/27; A61K 2800/651; A61K 2800/58; A61K 2800/28; A61K 2800/61; A61K 2800/56

(57) ABSTRACT

Described herein are oral care compositions comprising an orally acceptable carrier; an abrasive system comprising a calcium-based abrasive; and a core shell silica particle comprising; a metal silicate; and a silica particle comprising a core having a surface; wherein the metal silicate is chemically bound to a surface of the silica core; and wherein the metal silicate comprises a silicate of a divalent metal ion. Methods of making and using these compositions are also described.

### BACKGROUND

Calcium-based abrasive systems are cost-effective and preferred in many parts of the world; in particular, parts of Asia and Central and South Americas. For many years, these technologies have been a low-cost and effective way to fight cavities. However, tartar control has remained a challenge for oral care compositions comprising calcium-based abrasive systems.

As such, there is a need for low-cost compositions having superior anti-cavity efficacy, which also provide—inter alia—an anti-tartar benefit and a desirable taste profile.

Embodiments of the present invention are designed to meet these, and other, needs.

### BRIEF SUMMARY

Some embodiments of the present invention provide an oral care composition comprising: an orally acceptable carrier; an abrasive system comprising a calcium-based abrasive; and a core shell silica particle comprising; a metal silicate; and a silica particle comprising a core having a surface; wherein the metal silicate is chemically bound to a surface of the silica core; and wherein the metal silicate comprises a silicate of a divalent metal ion.

Other embodiments of the present invention provide oral care compositions comprising: an orally acceptable carrier; from about 5 wt. % to about 50 wt. % of a calcium-based abrasive system; and a core shell silica particle comprising: a metal silicate; and a silica particle comprising a core having a surface; wherein the metal silicate is chemically bound to a surface of the silica core; wherein the metal silicate comprises a silicate of a Zn²⁺; and wherein the total zinc level is from about 0.1 wt. % to about 1 wt. %, of the oral care composition.

Still further embodiments provide methods of treating, preventing, or ameliorating a symptom associated with, a disease, disorder or condition of the oral cavity, comprising administering a composition according to any foregoing claim to an oral surface of a mammal in need thereof.

Yet other embodiments provide for the use of any one of the compositions described herein in the manufacture of an oral care composition for treating, preventing, or ameliorating a symptom associated with, a disease, disorder or condition of the oral cavity.

Further areas of applicability of the present invention will become apparent from the detailed description provided hereinafter. It should be understood that the detailed description and specific examples are intended for purposes of illustration only and are not intended to limit the scope of the invention.

### BRIEF DESCRIPTION OF THE DRAWINGS

FIG. 1 depicts anti-cavity results for a comparative composition and an exemplary composition of the present invention.

FIG. 2 depicts anti-cavity results for three exemplary compositions of the present invention and a comparative composition

FIG. 3 depicts the results of a pH cycling test involving an exemplary composition of the present invention and comparative compositions.

FIG. 4 depicts the results of a pH cycling test involving exemplary compositions of the present invention and a comparative composition.

FIG. 5 depicts the results of a pH cycling test involving exemplary compositions of the present invention with a lower ratio of zinc and two comparative compositions.

FIG. 6 depicts the results of a Planktonic Assay involving three exemplary compositions of the present invention and two comparative compositions.

FIG. 7 depicts the results from anti-bacterial evaluations based on the University of Manchester model.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range, and for describing sub-ranges within the range. Any value within the range can be selected as the upper terminus of the sub-range. Any value within the range can be selected as the lower terminus of the sub-range.

In addition, all references, books, patents, and patent application publications cited herein are hereby incorporated by reference in their entireties. In the event of a conflict in a definition in the present disclosure and that of a cited reference, book, patent, or patent application publication, the present disclosure controls.

Unless otherwise specified, reference to ambient or room temperature refers to a temperature range of 20-25° C.

Unless otherwise specified, all percentages and amounts expressed herein and elsewhere in the specification should be understood to refer to percentages by weight based on the total weight of the composition.

The phrase “and/or” as used herein, with option A and/or option B for example, encompasses the individual embodiments of (i) option A; (ii) option B; and (iii) option A plus option B.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group and all possible subgroups of the main group, but also the main group absent one or more of the group members. The present invention also envisages the explicit exclusion of one or more of any of the group members in the claimed invention.

All combinations of the various elements described herein are within the scope of the invention unless otherwise indicated herein or otherwise clearly contradicted by context.

In some embodiments, the present invention provides oral care compositions comprising: an orally acceptable carrier; an abrasive system comprising a calcium-based abrasive system; and a core shell silica particle comprising; a metal silicate; and a silica particle comprising a core having a surface; wherein the metal silicate is chemically bound to a surface of the silica core; and wherein the metal silicate comprises a silicate of a divalent metal ion. In some embodiments, the calcium-based abrasive comprises an abrasive selected from: natural calcium carbonate; precipitated calcium carbonate; dicalcium phosphate, and calcium pyrophosphate.

In some embodiments, the divalent metal ion is selected from: Ca²⁺; Mg²⁺; Zn²⁺; Sn²⁺; Sr²⁺; Fe²⁺; Mo²⁺; Co²⁺; Ni²⁺; Mn²⁺; Cu²⁺; Pd²⁺; Mo²⁺; Ru²⁺; and a combination of two or more thereof. In other embodiments, the divalent metal ion is selected from Zn²⁺ and Sn²⁺. Still further embodiments provide oral care compositions wherein the divalent metal ion is Zn²⁺.

In some embodiments, the metal silicate further comprises a monovalent metal ion. In some embodiments, the monovalent metal ion is selected from Na⁺ and K⁺.

In certain embodiments, the silica is selected from the group consisting of a precipitated silica, a fumed silica, heat treated silica and a fused silica.

Pyrogenic silica (sometimes called fumed silica or silica fume) is a very fine particulate or colloidal form of silicon dioxide. It is prepared by burning SiCl₄ in an oxygen rich hydrocarbon flame to produce a “smoke” of SiO₂. The silica particles fuse with one another to form branched, three-dimensional chain-like aggregates as shown below:

SiCl₄+2H₂+O₂→SiO₂+4HCl.

Amorphous silica, silica gel, is produced by the acidification of solutions of sodium silicate. An initially formed gelatinous precipitate is then washed and then dehydrated to produce colorless microporous silica. Idealized equation involving a trisilicate and sulfuric acid is shown:

Na₂Si₃O₇+H₂SO₄→3SiO₂+Na₂SO₄+H₂O

In the majority of silicates, the Si atom shows tetrahedral coordination, with 4 oxygen atoms surrounding a central Si atom. The most common example is seen in the quartz crystalline form of silica SiO₂. In each of the most thermodynamically stable crystalline forms of silica, on average, all 4 of the vertices (or oxygen atoms) of the SiO₄ tetrahedra are shared with others, yielding the net chemical formula: SiO₂. SiO₂ has a number of distinct crystalline forms (polymorphs) in addition to amorphous forms. With the exception of stishovite and fibrous silica, all of the crystalline forms involve tetrahedral SiO₄ units linked together by shared vertices in different arrangements.

Precipitated silica includes, but is not limited to Zeodent® 114 and Zeodent® 165 (precipitated silica particles produced by J. M. Huber—synthetic amorphous silica), Sylodent® 783 produced by W.R. Grace, Sorbosil® AC-43 produced by Ineos (PQ Corp.)

The silica may be a fumed silica, such as Aerosil 200, produced by Evonik.

In another embodiment, the silica is a fused silica, which includes but is not limited to CAB-O-SIL® HP-60, produced by Cabot Corporation, TECO-SIL® 10 and TECO-SIL® 44css, produced by C-E Minerals, and Spheron P1500 made by the Japanese Glass Co.

In some embodiments, the core shell silica particle comprises a plurality of metal silicate layers. In some embodiments, the number of metal silicate layers may be from about 2 to about 100, about 2 to about 40, about 2 to about 12 or about 12 to about 40 layers. In other embodiments, the core shell silica particle may comprise 2, 4, 16, 32, 36 or 64 metal silicate layers.

In a preferred embodiment the silicate of the second metal ion comprises ZnSiO₃.xH₂O, wherein x is from 0 to 10.

In one embodiment the surface of the silica core is the outer surface of the silica core. In addition or as an alternative the surface of the silica core may be an internal surface of the silica core.

In some embodiments, the metal silicate comprising a divalent metal ion comprises at least about 30 wt. %, at least about 40 wt. %, at least about 50 wt. %, at least about 60 wt. %, at least about 70 wt. %, at least about 80 wt. %, or at least about 90 wt. %, of the total metal silicate of the core shell silica particle.

In some embodiments, the outer 10 nm depth of the core shell silica particle may comprise from 0.1 to 10 weight % metal silicate. In some embodiments the outer 10 nm depth of the core shell silica particle has the general formula:

(SiO₂)ₚ[O*ₒNₙ⁺Mₘ²⁺Uᵤ³⁺Vᵥ⁴⁺Hₕ⁺].qH₂O

wherein O* is oxygen in the silicate form; N is a monovalent metal ion; M is a divalent metal ion; U is a trivalent metal ion; V is a tetravalent metal ion; p, o, n, m, u, v, h and q are the atomic percentages of each component; and the total charge of each core shell silica particle is zero.

The atomic percentage for each component except H+ is typically determined by electron spectroscopy for chemical analysis (ESCA). In one example, using ESCA data, the following elements were detected:

O_{56.81}Si_{26.52}O*_{7.35}Na_{3.18}Zn_{4.65}Cl_{1.49}

By setting the total electric charge to zero by adding H+ and water, we conclude that in one embodiment the outer 10 nm depth of each particle may have the following composition:

(SiO₂)_{26.52}[O*_{7.35}Na_{3.18}Zn_{4.65}Cl_{1.49}H_{3.73}].3.77H₂O

The d(0.5) value of the particles is typically from 5 nm to 50 μm.

The d(0.5) value of the particles may be from 26 μm to 40 μm. Particles having a d(0.5) value within this range are typically opaque. Translucent particles are those which allow light to pass through, although it is not possible to see an image through the particles. This is distinguished from transparent compositions which allow light to pass through and an image can be seen through the composition. Methods for determining particle size are well known in the art. For example particle size may be determined using light scattering methodologies, such as using the Mastersizer 2000, Hydro 2000S, Malvern Instruments Limited.

The d(0.5) value of the particles may be from 18 μm to 25 μm. Particles having a d(0.5) value within this range are typically opaque.

The d(0.5) value of the particles may be from 10 μm to 15 μm. Particles having a d(0.5) value within this range are typically opaque.

In another embodiment, the d(0.5) value of the CSS particles may be from 5 μm to 15 μm.

In another embodiment, the d(0.5) value of the CSS particles may be from 2.5 μm to 4.5 μm.

In another embodiment, the d(0.5) value of the CSS particles may be from 5 nm to 20 nm.

In another embodiment, the d(0.5) value of the CSS particles may be from 10 nm to 15 nm.

In another embodiment, the d(0.5) value of the particles may be from 5 nm to 12 nm.

The d(0.5) or d50 of the particles is the diameter (typically in microns) that splits the distribution with half the population above and half below this diameter. The Dv50 (or Dv0.5) is the median for a volume distribution, Dn50 is used for number distributions, and Ds50 is used for surface distributions. In the present context, d(0.5) will be used to refer to the median particle size for a volume distribution (Dv0.5).

The d(0.1) value of the particles is the diameter that splits the distribution with 10% of the population below and 90% above this diameter.

The d(0.9) value of the particles is the diameter that splits the distribution with 90% of the population below and 10% above this diameter.

A value used to describe the distribution width of the particle size distribution is the span: Span=(d(0.9)−d(0.1))/d(0.5)

The span of the core shell silica particles according to the present invention is typically from 1.5 to 3.

In a preferred embodiment, the CSS have a d(0.1) of from 10 to 13 μm, a d(0.5) of from 30 to 33 μm, and a d(0.9) of from 61 to 64 μm.

In another preferred embodiment, the CSS have a d(0.1) of from 6 to 9 μm, a d(0.5) of from 18 to 21 μm, and a d(0.9) of from 41 to 45 μm.

In a further preferred embodiment, the CSS have a d(0.1) of from 3 to 5 μm, a d(0.5) of from 11 to 14 μm, and a d(0.9) of from 33 to 36 μm.

In preferred embodiments, the d(0.5) value of the CSS particles is less than the mean diameter of a human dentin tubule. This allows the CSS particles to enter the dentin tubules, which may be exposed on damage to the protective enamel layer. In human teeth, dentin tubule mean diameter near the dentino-enamel junction is 0.9 μm, the middle section of the dentin tubule has a diameter of about 1.2 μm and near the pulp the diameter is about 2.5 μm.

In another embodiment of the invention, a silica source is selected to produce CSS particles which fits into the dentin tubule (e.g. Aerosil® 200—a fumed silica (synthetic amorphous silica) with a d(0.5) of 0.012 μm). In another embodiment of the invention, the d(0.5) value of the CSS particles is less than 0.9 μm. In still another embodiment of the invention, the CSS particle has a d(0.5) in the range of 0.010 μm—less than 0.9 μm. In another embodiment of the invention, the CSS particles of the invention can also plug, block holes in the enamel.

The present core shell silica particles have surprisingly high surface charge density and ion exchange capacity. In an embodiment, the core shell silica particles have a surface charge density of from 0.5 to 4.5 meq/g silica. In an embodiment, the core shell silica particles have surface charge density of from 2 to 3 meq/g silica. In an embodiment, the core shell silica particles have a surface charge density of 2.45-2.55 meq/g silica.

In an embodiment, the core shell silica particles have a charge, or ion-exchange capacity of, from 0.05 to 0.1 C/cm² surface area. In an embodiment, the core shell silica particles have a charge, or ion-exchange capacity, of from 0.085 to 0.095 C/cm² surface area. In an embodiment, the core shell silica particles have a charge, or ion-exchange capacity, of from 0.089 C/cm² surface area.

In an embodiment of Zn-CSS particles, the amount of zinc adsorbed to surface monolayers of the particles is less than 50% of the maximum ion-exchange capacity of the particle for divalent ions. In an embodiment, the amount of zinc adsorbed to surface monolayers of the particles is 30-35% of the maximum ion-exchange capacity of the particle for divalent ions. In an embodiment, the amount of zinc adsorbed to surface monolayers of the particles is 33% of the maximum ion-exchange capacity of the particle for divalent ions.

In a further aspect, the present invention provides an oral care composition comprising any one of the core shell silica particles described herein.

In one embodiment the composition comprises from 0.01 to 0.5 weight % soluble metal ions. The soluble metal ions may be zinc ions. One of the advantages of the CSS compositions of the present invention is that CSS particles complex with metal ions such that the concentration of free metal ions in solution is low. High concentrations of free metal ions, such as zinc ions can provide disadvantages, particularly for oral care compositions. For example, a high concentration of soluble zinc ions can lead to a poor taste profile for the composition.

In some embodiments, the oral care composition further comprises an orally acceptable carrier.

In an embodiment of the composition, the core shell silica particles comprise a range selected from the ranges consisting of 0.1% to 35 weight %, based on the weight of the composition. In another embodiment of the composition, the CSS particles are present in an amount from 0.1% to 1%. In another embodiment of the composition, the CSS particles are present in an amount from 0.5% wt. % to 20 wt. %, In another embodiment of the composition, the CSS particles are present in an amount from 1% wt. % to 10 wt. %.

In an embodiment, the metal salt is present at 0.01-3.0 weight % of the composition. In an embodiment, the metal salt is present at 0.1-1.5 weight % of the composition. In an embodiment, the metal salt is present at 0.1-1.0 weight %. In an embodiment, the metal salt is present at 0.1-0.75 weight %. In an embodiment, the metal salt is present at about 0.1%, about 0.2%, about 0.25%, about 0.5%, about 0.7%, about 0.75%, about 1%, about 1.25%, about 1.5%, about 1.75%, about or about 2%, by weight, of the oral care composition. In some embodiments, the metal salt is present at 1 weight % or 2 weight %. In an embodiment the metal salt is ZnCl₂ in an amount of from 0.1% to 2%, by weight, of the composition.

In some embodiments, the calcium-based abrasive system is present in an amount of from about 5 wt. % to about 50 wt. % of the oral care composition. In some embodiments, the calcium-based abrasive system is present in an amount of from about 10 wt. % to about 50 wt. % of the oral care composition. In some embodiments, the calcium-based abrasive system is present in an amount of from about 15 wt. % to about 50 wt. % of the oral care composition. In some embodiments, the calcium-based abrasive system is present in an amount of from about 20 wt. % to about 50 wt. % of the oral care composition. In some embodiments, the calcium-based abrasive system is present in an amount of from about 25 wt. % to about 50 wt. % of the oral care composition. In other embodiments, the calcium-based abrasive system is present in an amount of from about 30 wt. % to about 45 wt. %, of the oral care composition. In further embodiments, the calcium-based abrasive system is present in an amount of from about 30 wt. % to about 40 wt. %, of the oral care composition. In some embodiments, the calcium-based abrasive system is present in an amount of from about 25 wt. % to about 35 wt. % of the oral care composition. Still further embodiments provide oral care compositions wherein the calcium-based abrasive system is present in an amount of about 30 wt. %, of the oral care composition.

In some embodiments, the amount of soluble divalent metal ion in the oral care composition is less than about 500 ppm, about 400 ppm, about 300 ppm, or about 250 ppm. In other embodiments, the amount of soluble divalent metal ion in the oral care composition is about 200 ppm. In some embodiments, the reduced level of soluble divalent metal ion is responsible for the improved taste profile of the compositions described herein.

In certain embodiments, the oral care composition comprises a total zinc level of from about 0.1 wt. % to about 1 wt. %, of the oral care composition. In other embodiments, the oral care composition comprises a total zinc level of from about 0.2 wt. % to about 0.9 wt. %, of the oral care composition. In some embodiments, the oral care composition comprises a total zinc level of from about 0.3 wt. % to about 0.8 wt. %, of the oral care composition. In further embodiments, the oral care composition comprises a total zinc level of from about 0.4 wt. % to about 0.75 wt. %, of the oral care composition. Still other embodiments provide oral care compositions comprising a total zinc level of from about 0.1 wt. % to about 1 wt. %, of the oral care composition. Yet other embodiments provide oral care compositions comprising a total zinc level of from about 0.1 wt. % to about 0.7 wt. %, of the oral care composition. In further embodiments, the oral care composition comprises a total zinc level of from about 0.1 wt. % to about 0.5 wt. %, of the oral care composition.

In some embodiments, the amount of added water does not exceed 20 wt. %, of the oral care composition.

Still further embodiments provide oral care compositions comprising: an orally acceptable carrier; from about 5 wt. % to about 50 wt. % of a calcium-based abrasive system; and a core shell silica particle comprising: a metal silicate; and a silica particle comprising a core having a surface; wherein the metal silicate is chemically bound to a surface of the silica core; wherein the metal silicate comprises a silicate of a Zn²⁺; and wherein the total zinc level is from about 0.1 wt. % to about 1 wt. %, of the oral care composition.

In some embodiments, the precipitated silica, fumed silica, heat treated silica or fused silica has a first diameter. In some embodiments, the core shell silica particle has a second diameter. In some embodiments, the first diameter is greater than the second diameter. In some embodiments, the metal silicate layer has an uneven surface.

In some embodiments, the calcium-based abrasive system comprises a plurality of calcium compounds. In some embodiments, the calcium-based abrasive system comprises: natural calcium carbonate; and precipitated calcium carbonate. In some embodiments, the natural calcium carbonate and the precipitated calcium carbonate are present in a weight ratio range from about 1:2 to 2:1. In other embodiments, the natural calcium carbonate and the precipitated calcium carbonate are present in a weight ratio of about 1:1.

In some embodiments, the calcium-based abrasive systems described herein consist essentially of one or more calcium abrasives. In some embodiments, the calcium-based abrasive systems described herein consist of one or more calcium abrasives.

Some embodiments comprise an additional abrasive, e.g. a silica abrasive.

Further embodiments provide methods of treating, preventing, or ameliorating a symptom associated with, a disease, disorder or condition of the oral cavity, comprising administering any one of the compositions described herein to an oral surface of a mammal in need thereof. Still further embodiments provide for the use of any one of the compositions described herein in the manufacture of an oral care composition for treating, preventing, or ameliorating a symptom associated with, a disease, disorder or condition of the oral cavity. In some embodiments, the disease, disorder or condition of the oral cavity is selected from: erosion; caries; inflammation; excessive tarter; gingivitis; periodontitis; dentin hypersensitivity; discolored teeth; xerostomia; bleeding gums; plaque overgrowth; and malodor.

In another embodiment of the invention, the composition may take any dosage form useful for oral administration. In an embodiment, the composition is a solid, a paste, a gel, or a liquid.

Illustrative examples of these include, but are not limited to, a dentifrice, e.g., a toothpaste, dental gel, dental cream, or tooth powder; a mouthwash, mouth rinse, or mouth spray; an oral slurry or liquid dentifrice; a gum or other confectionary; a lozenge; dental floss or dental tape; a prophylaxis paste or powder; a mono- or multi-layer oral film or gel strip, e.g., tooth strips or breath strips, preferably using a biodegradable or orally consumable film or gel; functional film or gel flakes or functional milli-, micro-, or nano-particles; a film-forming composition comprising pre-gel(s) or pre-polymer(s), e.g., film-forming dentifrices, dental paints; a tooth hardener; or a coating on an oral, e.g., orthodontic, appliance or implant.

For solid dentifrices such as toothpastes, the amount of water in the composition is selected from an amount of from about 10 wt. % to about 40 wt. %, about 15 wt. % to about 35 wt. %, 20 wt. % to about 30 wt. %.

In some embodiments, the composition further comprises an anti-malodor agent. In an embodiment, the additional anti-malodor compound is a known odor-controlling agent. In addition, other metal-containing compounds, such as those of copper, stannous, bismuth, strontium; and succulents or other ingredients which increase salivary flow, act to wash away odors, are useful in the compositions described herein. Certain strong citrus-based flavorants, odor-absorption complexes, which entrap or adsorb malodor molecules are also useful in the claimed compositions. For example, Ordenone® has the ability to encapsulate malodor molecules such as mercaptans, sulfides and amines within its structure, as disclosed in, for example, U.S. Pat. No. 6,664,254. Odor-controlling actives suitable also include, but are not limited to, enzymes that can interrupt the process by which odors are created. For example, odor-blocking enzymes such as arginine deiminase, can be effectively formulated in the compositions of the invention. Also, molecules that effectively inhibit the bacterial production of malodor molecules can be used to control odor, for example agents that interfere with the bacterial enzymes cysteine desulfhydrase and/or methionine gamma-lyase. Odor-controlling actives suitable for odor blocking or as odor blockers, include but are not limited to agents that act by oxidizing or otherwise chemically reacting with malodor molecules, including peroxides, perchlorites, and reactive molecules with activated double bonds.

The carrier may include, but is not limited to water or other aqueous solvent systems.

The orally acceptable carrier may further comprise a humectant. Possible humectants are ethanol, a polyhydric alcohol, which includes, but is not limited to glycerin, glycol, inositol, maltitol, mannitol, sorbitol, xylitol, propylene glycol, polypropylene glycol (PPG), polyethylene glycol (PEG) and mixtures thereof, or a saccharide, which includes, but is not limited to fructose, glucose, sucrose and mixtures of saccharides (e.g. honey).

The oral care composition may further comprise an anti-bacterial agent, which is not the core shell silica particle described herein. The anti-bacterial agent may be triclosan (5-chloro-2-(2,4-dichlorophenoxy)phenol); 8-hydroxyquinoline and salts thereof, zinc and stannous ion sources such as zinc citrate, zinc sulphate, zinc glycinate, sodium zinc citrate, stannous fluoride, stannous monofluorophosphate and stannous pyrophosphate; copper (II) compounds such as copper (II) chloride, fluoride, sulfate and hydroxide; phthalic acid and salts thereof such as magnesium monopotassium phthalate; sanguinarine; quaternary ammonium compounds, such as alkylpyridinium chlorides (e.g., cetylpyridinium chloride (CPC), combinations of CPC with zinc and/or enzymes, tetradecylpyridinium chloride, and N-tetradecyl-4-ethylpyridinium chloride); bisguanides, such as chlorhexidine digluconate, hexetidine, octenidine, alexidine; halogenated bisphenolic compounds, such as 2,2′ methylenebis-(4-chloro-6-bromophenol); benzalkonium chloride; salicylanilide, domiphen bromide; iodine; sulfonamides; bisbiguanides; phenolics; piperidino derivatives such as delmopinol and octapinol; magnolia extract; thymol; eugenol; menthol; geraniol; carvacrol; citral; eucalyptol; catechol; 4-allylcatechol; hexyl resorcinol; methyl salicylate; antibiotics such as augmentin, amoxicillin, tetracycline, doxycycline, minocycline, metronidazole, neomycin, kanamycin and clindamycin; or mixtures thereof.

In some embodiments, the anti-bacterial agent is present at a concentration selected from the group consisting of from 0.001% to 3%, by weight, 0.05% to 2%, by weight and 0.075% to 1.5% by weight.

Alternatively, there is no additional anti-bacterial agent except for the core shell silica particles of the invention.

In some embodiments, the oral care composition may further include anti-caries agents, desensitizing agents, viscosity modifiers, diluents, surfactants, emulsifiers, foam modulators, pH modifying agents, abrasives, mouth feel agents, sweetening agents, flavor agents, colorants, preservatives, amino acids, anti-oxidants. anti-calculus agents, a source of fluoride ions, thickeners, an active agent for prevention or treatment of a condition or disorder of hard or soft tissue of the oral cavity, adhesive agents, a whitening agent and combinations thereof. It is understood that while general attributes of each of the above categories of materials may differ, there may be some common attributes and any given material may serve multiple purposes within two or more of such categories of materials. Preferably, the carrier is selected for compatibility with other ingredients of the composition.

Some embodiments of the present invention optionally comprise an amino acid. Suitable amino acids include, but are not limited to arginine, cysteine, leucine, isoleucine, lysine, alanine, asparagine, aspartate, phenylalanine, glutamate, glutamic acid, threonine, glutamine, tryptophan, glycine, valine, praline, serine, tyrosine, and histidine, and a combination of two or more thereof. The amino acids can include R- and L-forms and salt forms thereof. The amino acids (and salt forms thereof) can also include acid ester and/or fatty amide derivatives of the amino acid (e.g. ethyl lauroyl arginate hydrochloride (ELAH)).

An embodiment of the composition optionally comprises an antioxidant. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

An embodiment of the composition optionally comprises an anticalculus (tartar control) agent. Suitable anticalculus agents include without limitation phosphates and polyphosphates (for example pyrophosphates), polyaminopropanesulfonic acid (AMPS), hexametaphosphate salts, zinc citrate trihydrate, polypeptides, polyolefin sulfonates, polyolefin phosphates, diphosphonates. The anticalculus agent is present at about 0.1% to about 30%. The oral composition may include a mixture of different anticalculus agents. In one preferred embodiment, tetrasodium pyrophosphate (TSPP) and sodium tripolyphosphate (STPP) are used. The anticalculus agent comprises TSPP at about 1-2% and STPP at about 7% to about 10%.

An embodiment of the composition optionally comprises at least one orally acceptable source of fluoride ions. Any known or to be developed in the art may be used. Suitable sources of fluoride ions include fluoride, stannous fluoride, sodium fluoride, potassium fluoride, amine fluoride, ammonium fluoride, stannous monofluorophosphate, sodium monofluorophosphate, potassium monofluorophosphate, amine monofluorophosphate, ammonium monofluorophosphate, stannous fluorosilicate, sodium fluorosilicate, potassium fluorosilicate, amine fluorosilicate ammonium fluorosilicate, and mixtures thereof. One or more fluoride ion-releasing compound is optionally present in an amount providing a total of about 100 to about 20,000 ppm, about 200 to about 5,000 ppm, or about 500 to about 2,500 ppm, fluoride ions.

An embodiment of the composition optionally comprises various dentifrice ingredients to adjust the rheology and feel of the composition such as surface active agents, thickening or gelling agents, etc.

An embodiment of the composition optionally comprises a stannous ion or a stannous ion source. Suitable stannous ion sources include without limitation stannous fluoride, other stannous halides such as stannous chloride dihydrate, stannous pyrophosphate, organic stannous carboxylate salts such as stannous formate, acetate, gluconate, lactate, tartrate, oxalate, malonate and citrate, stannous ethylene glyoxide and the like. One or more stannous ion sources are optionally and illustratively present in a total amount of about 0.01% to about 10%, for example about 0.1% to about 7% or about 1% to about 5%.

An embodiment of the composition optionally comprises a surface active agent (surfactant). Suitable surfactants include without limitation water-soluble salts of C₈-C₂₀ alkyl sulfates, sulfonated monoglycerides of C₈-C₂₀ fatty acids, sarcosinates, taurates, sodium lauryl sulfate, sodium cocoyl monoglyceride sulfonate, sodium lauryl sarcosinate, sodium lauryl isoethionate, sodium laureth carboxylate and sodium dodecyl benzenesulfonate, and cocoamidopropyl betaine.

An embodiment of the composition optionally comprises a thickener. Any orally acceptable thickening agent can be used, including without limitation carbomers, also known as carboxyvinyl polymers, carrageenans, also known as Irish moss and more particularly—carrageenan (iota-carrageenan), high molecular weight polyethylene glycols (such as Carbowax®, available from The Dow Chemical Company), cellulosic polymers such as hydroxyethylcellulose, carboxymethylcellulose (CMC) and salts thereof, e.g., CMC sodium, natural gums such as karaya, xanthan, gum arabic and tragacanth, colloidal magnesium aluminum silicate, and colloidal and/or fumed silica and mixtures of the same. One or more thickening agents are optionally present in a total amount of about 0.1% to about 90%, for example about 1% to about 50% or about 5% to about 35%.

An embodiment of the composition optionally comprises flavorants, sweeteners, colorants, foam modulators, mouth-feel agents and others additively may be included if desired, in the composition.

An embodiment of the composition optionally comprises one or more further active material(s), which is operable for the prevention or treatment of a condition or disorder of hard or soft tissue of the oral cavity, the prevention or treatment of a physiological disorder or condition, or to provide a cosmetic benefit. Examples of such further active ingredient comprise a sialagogue or saliva-stimulating agent, an antiplaque agent, an anti-inflammatory agent, and/or a desensitizing agent.

Adhesion enhancing agents can also be added to the oral care compositions which include but is not limited to waxes, inclusive of bees' wax, mineral oil, plastigel, (a blend of mineral oil and polyethylene), petrolatum, white petrolatum, shellac, versagel (blend of liquid paraffin, butene/ethylene/styrene hydrogenated copolymer) polyethylene waxes, microcrystalline waxes, polyisobutene, polyvinyl pyrrolidone/vinyl acetate copolymers, and insoluble polyacrylate copolymers.

Also effective as adhesion enhancing agents are liquid hydrophilic polymers including polyethylene glycols, nonionic polymers of ethylene oxide having the general formula: HOCH₂ (CH₂OCH₂)ₙ₁CH₂OH wherein n1 represents the average number of oxyethylene groups. Polyethylene glycols available from Dow Chemical are designated by a number such as 200, 300, 400, 600, 2000 which represents the approximate average molecular weight of the polymer, as well as nonionic block copolymer of ethylene oxide and propylene oxide of the formula: HO(C₂H₄O)ₐ₁(C₃H₆O)_{b1}(C₂H₄O)_{c1}H. The block copolymer is preferably chosen (with respect to a1, b1 and c1) such that the ethylene oxide constituent comprises from about 65 to about 75% by weight, of the copolymer molecule and the copolymer has an average molecular weight of from about 2,000 to about 15,000 with the copolymer being present in the liquid tooth whitening composition in such concentration that the composition is liquid at room temperatures.

A particularly desirable block copolymer for use in the practice of the present invention is available commercially from BASF and designated Pluraflo L1220 (PEG/PPG 116/66) which has an average molecular weight of about 9,800. The hydrophilic poly(ethylene oxide) block averages about 65% by weight of the polymer.

Synthetic anionic polycarboxylates may also be used in the oral compositions of the present invention as an efficacy enhancing agent for any antibacterial, antitartar or other active agent within the dentifrice composition. Such anionic polycarboxylates are generally employed in the form of their free acids or preferably partially or more preferably fully neutralized water soluble alkali metal (e.g. potassium and preferably sodium) or ammonium salts. Preferred are 1:4 to 4:1 copolymers of maleic anhydride or acid with another polymerizable ethylenically unsaturated monomer, preferably methylvinylether/maleic anhydride having a molecular weight (M.W.) of about 30,000 to about 1,800,000 most preferably about 30,000 to about 700,000. Examples of these copolymers are available from GAF Corporation under the trade name GANTREZ® (methylvinylether/maleic anhydride), e.g., AN 139 (M.W. 500,000), AN 119 (M.W. 250,000); S-97 Pharmaceutical Grade (M.W. 700,000), AN 169 (M.W. 1,200,000-1,800,000), and AN 179 (M.W. above 1,800,000); wherein the preferred copolymer is S-97 Pharmaceutical Grade (M.W. 700,000).

When present, the anionic polycarboxylates is employed in amounts effective to achieve the desired enhancement of the efficacy of any antibacterial, antitartar or other active agent within the oral composition. Generally, the anionic polycarboxylates is present within the oral composition from about 0.05% to about 4% by weight, preferably from about 0.5% to about 2.5% by weight.

Some embodiments provide a composition comprising a humectant comprising sorbitol; a high cleaning silica abrasive; a thickening system comprising fumed silica and carrageenan; a combination of precipitated calcium carbonate and natural calcium carbonate; potassium hydroxide; less than 30 wt. % water. In some embodiments, the thickening system comprises less than 2 wt. % of the composition. In some embodiments, the thickening system comprises less than 1.5 wt. % of the composition. In some embodiments, the thickening system comprises less than 1 wt. % of the composition.

Adhesion enhancing agents employed in compositions of various embodiments of the invention are present in an amount of from about 0 to about 20% by weight. Preferably, the adhesion enhancing agents are present in an amount of from about 2 to about 15% by weight.

An embodiment of the composition optionally comprises a whitening agent which includes, but is not limited to peroxide compounds such as hydrogen peroxide, peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof. In various embodiments, the peroxide compound comprises hydrogen peroxide, urea peroxide, sodium percarbonate and mixtures thereof.

In some embodiments a non-peroxide whitening agent may be provided. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite, pigments or dyes. In some embodiments the whitening agent is separated from the aqueous carrier. In some embodiments the whitening agent is separated from the aqueous carrier by encapsulation of the whitening agent.

In one embodiment of the composition, the composition comprises about 65%-99.9% of the carrier and further included ingredients, i.e. one or more of anti-caries agents, desensitizing agents, viscosity modifiers, diluents, surfactants, emulsifiers, foam modulators, pH modifying agents, abrasives, mouth feel agents, sweetening agents, flavor agents, colorants, preservatives, amino acids, anti-oxidants, anti-calculus agents, a source of fluoride ions, thickeners, an active agent for prevention or treatment of a condition or disorder of hard or soft tissue of the oral cavity, a whitening agent and combinations thereof. In another embodiment of the composition, the composition comprises about 80%-99.5% of the carrier and further included ingredients. In another embodiment of the composition, the composition comprises about 90%-99% of the carrier and further included ingredients.

The description of the optional ingredients above is also intended to include any combination of ingredients.

In some embodiments, the core shell silica particles described herein may be prepared in accordance with the processes described in US 2016/0338920 or US 2016/0338919, the contents of which are hereby incorporated herein in their entireties.

In an embodiment, the silica used can be any abrasive silica. The silica may be selected from the group consisting of a precipitated silica, a fumed silica and a fused silica.

Precipitated silica includes, but is not limited to Zeodent® 114 and Zeodent® 165 (precipitated silica particles produced by J.M. Huber—chemical name: synthetic amorphous silica), Sylodent® 783 produced by W.R. Grace, Sorbosil® AC-43 produced by Ineos (PQ Corp.)

The silica may be a fumed silica, such as Aerosil 200, produced by Evonik.

In another embodiment, the silica is a fused silica, which includes but is not limited to CAB-O-SIL® HP-60, produced by Cabot Corporation, TECO-SIL® 10 and TECO-SIL® 44css, produced by C-E Minerals, and Spheron P1500 made by the Japanese Glass Co.

Suitable silicas for use in the invention also include colloidal silicas (thickening silicas) having, such as the aerogels Syloid 244 and 266 (available from W. R. Grace Company), Aerosil (available from DeGussa Co.) and pyrogenic silicas sold under the tradename Cab-O-Sils (available from Cabot Corporation). Tixosil 333 and Tixosil 43B (available from Rhodia Ltda.), Zeodent 165 (available from J. M. Huber Corporation).

Other suitable silicas for use in the invention include silica abrasives which in turn include silica gels and precipitated amorphous silicas. These silicas are colloidal particles/particulates having an average particle size ranging from about 3 microns to about 12 microns, and more preferably between about 5 to about 10 microns and a pH range from 4 to 10 preferably 6 to 9 when measured as a 5% by weight slurry. Illustrative of silica abrasives useful in the practice of the present invention are marketed under the trade designation Sylodent XWA by Davison Chemical Division of W.R. Grace & Co., Baltimore, Md. 21203. Sylodent 650 XWA, a silica hydrogel composed of particulates of colloidal silica having a water content of 29% by weight averaging from about 7 to about 10 microns in diameter.

Other types of silica abrasives suitable for use in the invention include precipitated silicas having a mean particle size of up to about 20 microns, such as Zeodent 115, marketed by J.M. Huber Chemicals Division, Havre de Grace, Md. 21078, or Sylodent 783 marketed by Davison Chemical Division of W.R. Grace & Company.

An average depth of from 1 to 15 nm of silica may be removed from the surface of the silica particle to form the silica core, and metal silicate is formed on top of the silica core. The average depth of silica removed typically increases as the weight ratio for the amount of base to the amount of silica particles increases. The d(0.5) of the silica core may be from 1 to 15 nm less than the d(0.5) of the silica particles of the starting material. The d(0.5) of the silica core may be about 2 nm less than the d(0.5) of the silica particles of the starting material. The d(0.5) particle diameter of the silica core may be about 6 nm less than the d(0.5) of the silica particles of the starting material. There is a greater percentage reduction in particle diameter for rigid silica particles such as fumed silica than for porous silica particles such as high cleaning silica. For example, for fumed silica the percentage reduction in particle diameter (d(0.5)) may be approximately 15%, whilst for porous high cleaning silica the percentage reduction in particle diameter (d(0.5)) may be approximately 0.06%.

The formation of the core shell silica particles of the invention described above can be effected by manipulating the amount of based used, the amount of humectant used, the amount of metal salt used, and varying the temperature of the reaction.

In an embodiment, the end point of the process results when the d(0.5) value of the core shell silica particles formed by the process is at least 5% greater in diameter than the d(0.5) value of the silica (SiO₂) starting material. In another embodiment, the core shell silica particle is from 5%-10% greater in diameter than the average particle diameter of the silica starting material.

The core shell silica particles formed may comprise from 0.0 to 0.5 weight % soluble metal ions. The soluble metal ions are preferably soluble zinc ions. As discussed above a low concentration of soluble metal ions, i.e. a low concentration of free metal ions such as zinc ions which can form a complex with the CSS can be used to prepare oral care compositions with an improved taste profile.

The formation of the core shell particles can also be monitored by determining the conductivity of the reaction mixture. The end point of the process results when the conductivity of the reaction mixture decreases by at least 250 micro Siemens/cm (μS/cm) because the electric charges transfer from highly mobile ions (NaOH) to much less mobile silica surface (mobility≈0). In yet another embodiment, the end point of the process results when the conductivity of the reaction mixture decreases by 250-400 μS/cm. Typically, the core shell silica particles are formed when the conductivity of the reaction mixture decreases by at least 2 milliSiemens/cm (mS/cm). Usually, the core shell silica particles are formed when the conductivity of the reaction mixture decreases by at least 5 mS/cm.

In some embodiments, the compositions described herein further comprise an unbound silicate comprising a divalent metal ion (e.g., Zn²⁺).

In a further aspect, the present invention provides a method of reducing or eliminating malodor in the oral cavity of a patient in need thereof, which comprises applying to the oral surfaces of the patient an oral care composition as defined above.

In one embodiment of the method, the patient is a mammal, which includes, but is not limited to humans and animals (e.g. dogs, cats, horses, cattle, sheep, llamas, etc.).

Embodiments of the present invention are further described in the following examples. The examples are merely illustrative and do not in any way limit the scope of the invention as described and claimed.

### EXAMPLES

### Example 1

Table 1 (below) describes an exemplary composition of the present invention (Ex. 1), along with a comparative composition (Comp. Ex. 1) which does not contain the inventive combination of ingredients.

TABLE 1

| Ingredients | Comp. Ex. 1 | Ex. 1 |
|---|---|---|
| Potassium hydroxide | — | 3.66 |
| Silica Abrasive | — | 5 |
| Sorbitol | 33.00 | 33.00 |
| Carrageenan | 0.825 | 0.825 |
| Sodium Saccharin | 0.27 | 0.27 |
| Sodium MFP | 0.76 | 0.76 |
| Sodium Bicarbonate | 0.50 | 2.50 |
| Water | 15.85 | 19.24 |
| Sodium Silicate | 1.00 | — |
| Thickening Silica | 1.75 | 0.00 |
| NCC | 19.50 | 14.00 |
| PCC | 21.50 | 16.00 |
| Triclosan | 0.30 | — |
| Titanium Dioxide | 1.00 | 1.00 |
| SLS Granules | 2.50 | 2.50 |
| Flavor | 0.95 | 0.95 |
| Benzyl Alcohol | 0.30 | 0.30 |
| TOTAL | 100 | 100 |

### Example 2

Toothpaste/CaCl₂ slurries are prepared to investigate the calcium chelation capabilities of of the formulas. Five grams (5 g) of toothpaste is mixed with about forty grams (40 g) of water. CaCl₂2.H₂O is added to the slurry as the calcium source. Sample 1 and Sample 2 are adjusted to the same pH by adding HCl. pH adjustment was done for Samples 3 and 4, as well. The slurries are well mixed then centrifuged. The supernatants are evaluated for levels of soluble calcium. The results of these evaluations are shown in Table 2 (below).

TABLE 2

| | Soluble | Soluble | | |
|---|---|---|---|---|
| | Ca²⁺ | Ca²⁺ | Chelated | Chelated |
| | Theoretical | Experimental | Ca²⁺ | Ca²⁺ |
| Samples* | Ppm | | | % |
| Sample 1 | 328 | 266 | 62 | 19% |
| Sample 2 | 327 | 143 | 184 | 56% |
| Sample 3 | 985 | 823 | 162 | 16% |
| Sample 4 | 980 | 491 | 489 | 50% |

Sample 1: 5 g Comp. Ex. 1 + 0.05 g CaCl₂ 2•H₂O + 40.1 g H₂O
Sample 2: 5 g Ex. 1 + 0.05 g CaCl₂ 2•H₂O + 40.1 g H₂O
Sample 3: 5 g Comp. Ex. 1 + 0.15 g CaCl₂ 2•H₂O + 40.1 g H₂O
Sample 4: 5 g Ex. 1 + 0.15 g CaCl₂ 2•H₂O + 40.1 g H₂O

As shown in Table 2 (above), the theoretical soluble calcium for Samples 1 and 2, is about 328 ppm. However, the experimental soluble calcium in Sample 1 is 266 ppm, which means it chelated 62 ppm (19%) calcium; whereas the experimental soluble calcium in Sample 2 (an exemplary composition of the present invention) is 143 ppm, which means it chelated 184 ppm (56%) calcium—three times the chelation provided by a comparative composition (Sample 1). For Samples 3 and 4, the theoretical soluble calcium is about 980 ppm; and the chelation results from these Samples show the same trend. Specifically, an exemplary composition of the present invention (Sample 4) chelated fifty percent (50%) of the calcium ions; again, more than three times the chelation provided by the comparative composition (Sample 3).

The data described in Table 2 (above) demonstrates that compositions of the present invention provide an unexpected, ability to chelate calcium, which correlates with a far superior tartar control benefit.

### Example 3

Two saliva based pH cycling tests are conducted on four (4) exemplary compositions of the present invention (Ex. 4 to Ex. 7) and two comparative compositions (Comp. Ex. 4 and Comp. 5). The pH cycling tests may be performed in accordance with methods generally known to those skilled in art. It is believed this saliva-based test mimics the environment of the oral cavity, because the buffer and acid solutions used in the test are prepared with human saliva. In addition, the anti-cavity influence from fluoride is accounted for in the results.

Table 3 (below) provides the formula details for the compositions evaluated in the aforementioned pH cycling test.

TABLE 3

| | | | | | Comp. | Comp. |
|---|---|---|---|---|---|---|
| | Ex. 4 | Ex. 5 | Ex. 6 | Ex. 7 | Ex. 4 | Ex. 5 |
| Ingredient | Wt. % | | | | | |
| Calcium | 36 | 30 | 30 | 30 | 41 | 41 |
| Abrasive(s) | | | | | | |
| Humectant(s) | 23 | 31 | 33 | 33 | 33 | 33 |
| Sodium Silicate | — | — | — | — | 1 | 1 |
| Water | 23.235 | 22.235 | 19.235 | 19.535 | 15.145 | 15.145 |
| Fluoride | 0.76 | 0.76 | 0.76 | 0.76 | 0.76 | 0.76 |
| Source(s) | | | | | | |
| Tetrapotassium | — | — | — | — | 1 | 1 |
| pyrophosphate | | | | | | |
| Preservative(s) | 0.3 | 0.3 | 0.3 | 0.3 | 0.3 | 0.3 |
| Surfactant(s) | 2.5 | 2.5 | 2.5 | 2.5 | 2.5 | 2.5 |
| Thickener(s) | 0.825 | 0.825 | 0.825 | 0.825 | 2.575 | 2.575 |
| Minors | 2.72 | 2.72 | 2.72 | 2.72 | 2.72 | 2.72 |
| Zinc ion | 1 | 0.5 | 0.25 | 0.1 | — | — |
| source(s) | | | | | | |
| Silica | 5 | 5 | 5 | 5 | — | — |
| Abrasive(s) | | | | | | |
| Potassium | 3.66 | 3.66 | 3.66 | 3.66 | — | — |
| hydroxide | | | | | | |

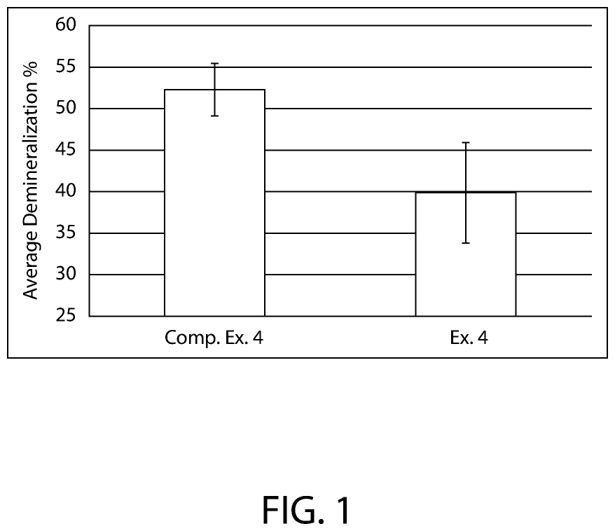

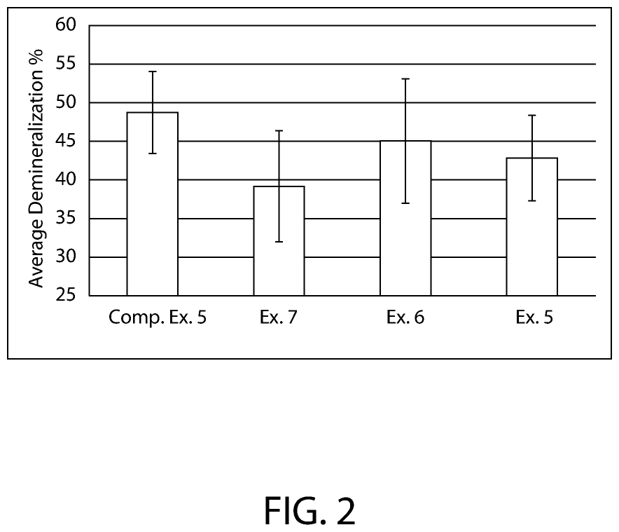

The results of the pH cycling tests are described in FIGS. 1 and 2. FIG. 1 shows the anti-cavity results of a comparative composition (Comp. Ex. 4) and an exemplary composition of the present invention containing zinc-core shell silica (Zn-CSS) particles of the present invention with one percent (1%) zinc. These results demonstrate that the inventive combination of the present invention provides a superior anti-cavity benefit to a substantially similar comparative composition. Specifically, the average demineralization observed with the comparative composition (Comp. Ex. 4) is 52%, whereas the average demineralization observed with the exemplary composition of the present invention (Ex. 4) is 40%. This unexpected reduction in demineralization provided by an exemplary composition of the present invention, is not insignificant.

FIG. 2 describes data confirming a similar benefit with three additional exemplary compositions of the present invention (Ex. 5 to Ex. 7), as compared to another substantially similar comparative composition (Comp. Ex. 5). As illustrated by FIG. 2, the three exemplary compositions of the present invention provide a significant improvement over the comparative composition. In particular, Ex. 7 containing 0.1% zinc provided an average demineralization of only 39%, as compared to the Comp. Ex. 5, which provided an average demineralization of about 49%.

### Example 4

Three additional pH cycling tests were performed on exemplary compositions of the present invention and comparative compositions. The pH cycling tests were performed in accordance with the methods described hereinabove in Example 3. Table 4 (below) provides the formula details for the compositions evaluated in these additional pH cycling tests.

TABLE 4

| | | | | Comp. | Comp. | Comp. |
|---|---|---|---|---|---|---|
| | Ex. 8 | Ex. 9 | Ex. 10 | Ex. 6 | Ex. 7 | Ex. 8 |
| Ingredient | Wt. % | | | | | |
| Calcium | 25 | 30 | 30 | 36 | 31 | 41 |
| Abrasive(s) | | | | | | |
| Humectant(s) | 31 | 31 | 31 | 33 | 23 | 33 |
| Sodium Silicate | — | — | — | 1 | — | 1 |
| Water | 28.13 | 21.95 | 21.65 | 13.895 | 27.235 | 15.145 |
| Fluoride | 0.76 | 0.76 | 0.76 | 0.76 | 0.76 | 0.76 |
| Source(s) | | | | | | |
| Tetrapotassium | — | — | — | 1 | 1 | 1 |
| pyrophosphate | | | | | | |
| Preservative(s) | 0.3 | 0.3 | 0.3 | 0.3 | 0.3 | 0.3 |
| Surfactant(s) | 2.5 | 2.5 | 2.5 | 2.5 | 2.5 | 2.5 |
| Thickener(s) | 0.6 | 0.61 | 0.41 | 2.575 | 0.825 | 2.575 |
| Minors | 2.72 | 2.72 | 2.72 | 2.72 | 2.72 | 2.72 |
| Zinc ion | 1.33 | 1.5 | 2 | 0.2 | — | 0.5 |
| source(s) | | | | | | |
| Silica | 5 | 5 | 5 | 5 | 5 | — |
| Abrasive(s) | | | | | | |
| Potassium | 3.66 | 3.66 | 3.66 | 1.25 | 3.66 | — |
| hydroxide | | | | | | |
| Calcium chloride | — | — | — | — | 2 | — |

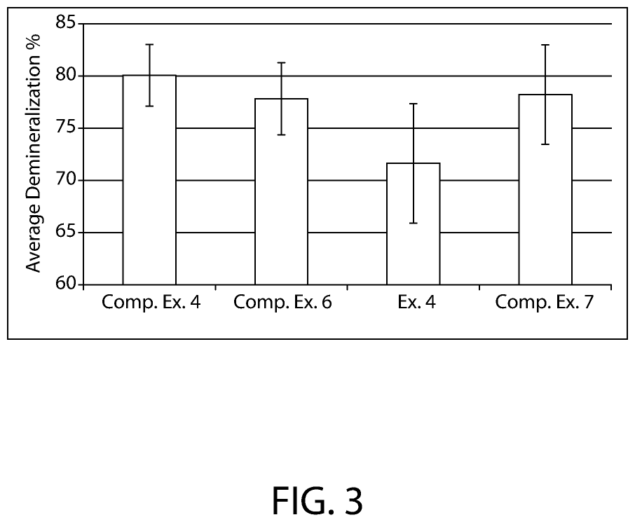

In the first pH cycling test, and exemplary composition of the present invention (Ex. 4) and three comparative compositions (Comp. Ex. 4, Comp. Ex. 6 and Comp. Ex. 7) are evaluated. As illustrated by the data depicted in FIG. 3, the exemplary composition of the present invention provided significantly less demineralization than the comparative compositions.

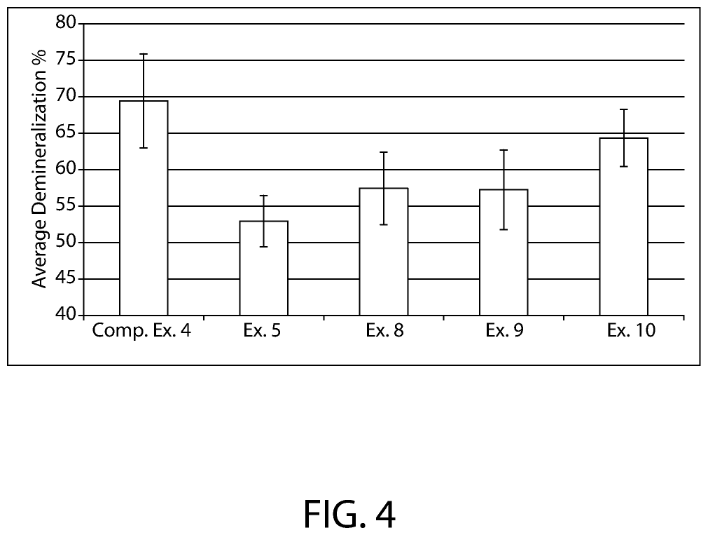

In a second pH cycling test, four exemplary compositions of the present invention (Ex. 5 and Ex. 8 to Ex. 10) and one comparative composition (Comp. Ex. 4) were evaluated. As illustrated by the data depicted in FIG. 4, exemplary compositions of the present invention provide a significant increase in protection against demineralization as compared to a comparative composition that does not contain the inventive combination of ingredients. This data also demonstrates that these benefits are provided by the compositions of the present invention having a range of zinc concentrations.

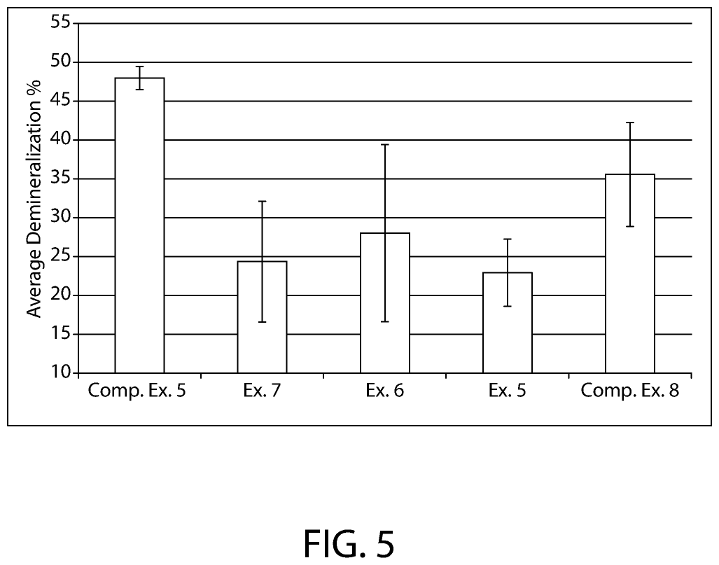

In a third pH cycling test, the anti-cavity efficacy of exemplary compositions of the present invention with a lower ratio of zinc (Ex. 5 to Ex. 7) was compared with the anti-cavity efficacy of two comparative compositions (Comp. Ex. 5 and Comp. Ex. 8). As illustrated by the data depicted in FIG. 5, the exemplary compositions of the present invention once again outperformed the comparative compositions. This data is even more surprising when read in light of the results from Comp. Ex. 8, which contains 0.5% zinc, but not in accordance with the compositions of the present invention (see, e.g. Ex. 5 vs. Comp. Ex. 8). The inventiveness of the compositions of the present invention is underscored by these results, which—at a minimum—demonstrate that the anti-cavity efficacy of zinc can be significantly enhanced through delivery via silicate groups.

### Example 5

The Planktonic Assay is used as a quick test method to determine the efficacy of bioactive metal compound in a formula. This assay uses a mixed species bacterial inoculum and the metabolic indicator dye Resazurin is used as a measure of bacterial viability following treatment. A five species mix of planktonic bacteria (Actinomyces viscosus, Lactobacillus casei, Streptococcus oralis, Veilonella parvula and Fusobacterium nucleatum) is treated for about 1 hour with the indicated dilution of dentifrice. Following treatment, the samples are washed and incubated with the non-fluorescent blue dye Resazurin. When metabolically active cells reduce Resazurin, it is converted to the pink fluorescent dye resorufin. By comparing the fluorescence of the test cultures to a standard curve, we can determine the percentage of the initial population of bacteria that remains viable after the 1 h treatment. For this study, the test treatment was run at low concentration (1:250) of dentifrice. It is necessary to use such low concentrations of dentifrice in order to minimize the effects of surfactants and other excipients found in toothpaste formulas, which can confound studies done on more concentrated samples. Three exemplary compositions of the present invention (Ex. 11 to Ex. 13) and two comparative compositions were evaluated. The formula details for the three comparative compositions are provided below in Table 5. The comparative compositions were a triclosan containing commercial toothpaste and Comp. Ex. 4 (described above in Table 3).

| | Ex. 11 | Ex. 12 | Ex. 13 |
|---|---|---|---|
| Ingredient | Wt. % | | |
| Calcium Abrasive(s) | 30 | 30 | 30 |
| Humectant(s) | 33 | 33 | 33 |
| Sodium Silicate | — | — | — |
| Water | 21.015 | 21.995 | 22.495 |
| Fluoride Source(s) | 0.76 | 0.76 | 0.76 |
| Tetrapotassium pyrophosphate | — | — | — |
| Preservative(s) | 0.3 | 0.3 | 0.3 |
| Surfactant(s) | 2.5 | 2.5 | 2.5 |
| Thickener(s) | 0.705 | 0.725 | 0.825 |
| Minors | 2.72 | 2.72 | 2.72 |
| Zinc ion source(s) | 1 | 0.5 | 0.1 |
| Silica Abrasive(s) | 5 | 5 | 5 |
| Potassium hydroxide | 2 | 2 | 2 |
| Chelant(s) | — | — | 0.2 |

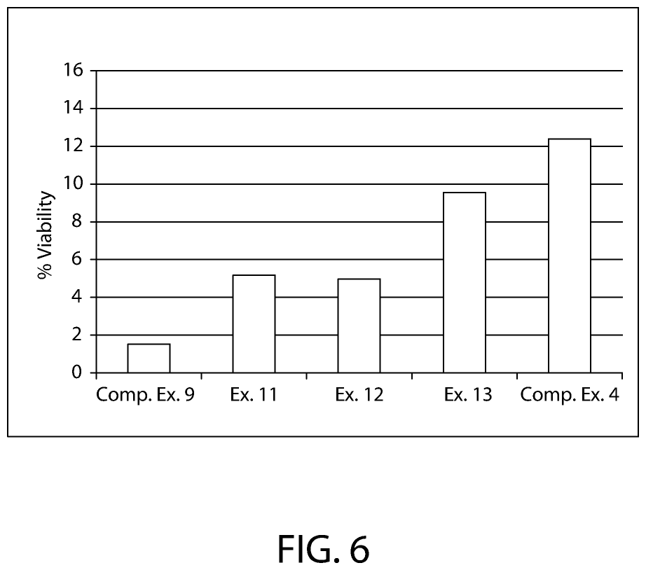

The results of the Planktonic Assay are described in FIG. 6.

### Example 6

The University of Manchester anaerobic model (UoM) is used to provide a more sensitive indication of potential efficacy of the three exemplary compositions of the present invention and two comparative compositions evaluated in Example 5 (above). In this model, saliva is collected from healthy volunteers and pooled together to be used as inoculum. Each sample was treated in triplicate twice a day for 8 to 10 days. Biofilm is recovered on day 10 (i.e. after 18 treatments) to measure total anaerobic bacteria in terms of ATP RLU vales. Results are expressed as Log RLU. Presence of ATP indicates viability of bacteria.

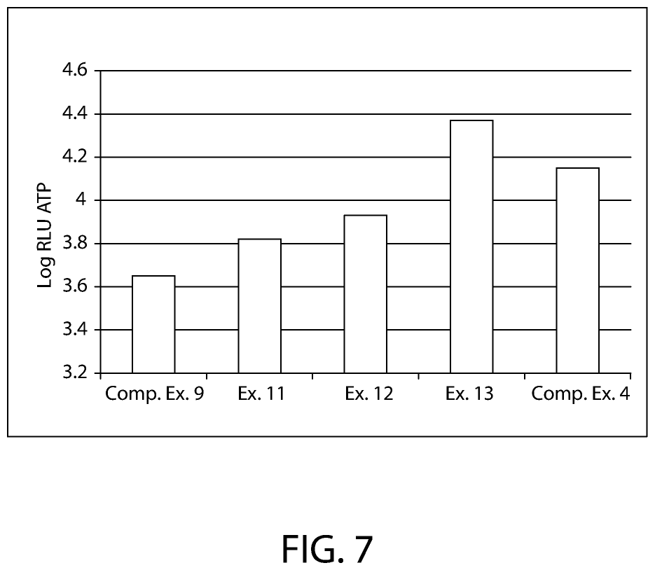

The data depicted in FIG. 7 illustrates the anti-bacterial results based on the University of Manchester model.

As those skilled in the art will appreciate, numerous changes and modifications may be made to the embodiments described herein without departing from the spirit of the invention. It is intended that all such variations fall within the scope of the appended claims.

1-39. (canceled)

40. An oral care composition comprising:
an orally acceptable carrier;
a calcium-based abrasive system; and
a core shell silica particle comprising;
a metal silicate; and
a silica particle comprising a core having a surface;
wherein the metal silicate is chemically bound to a surface of the silica core; and
wherein the metal silicate comprises a silicate of a divalent metal ion.

41. The oral care composition according to claim 40, wherein the calcium-based abrasive system comprises a compound selected from: natural calcium carbonate; precipitated calcium carbonate; dicalcium phosphate, and calcium pyrophosphate.

42. The oral care composition according to claim 40, wherein the divalent metal ion is selected from: Ca²⁺; Mg²⁺; Zn²⁺; Sn²⁺; Sr²⁺; Fe²⁺; Mo²⁺; Co²⁺; Ni²⁺; Mn²⁺; Cu²⁺; Pd²⁺; Mo²⁺; Ru²⁺; and a combination of two or more thereof.

43. The oral care composition according to claim 42, wherein the divalent metal ion is selected from Zn²⁺ and Sn²⁺.

44. The oral care composition according to claim 40, wherein the metal silicate further comprises a monovalent metal ion.

45. The oral care composition according to claim 44, wherein the monovalent metal ion is selected from Na⁺ and K⁺.

46. The oral care composition according to claim 40, wherein the core shell particles comprise a plurality of metal silicate layers.

47. The oral care composition according to claim 40, wherein the metal silicate comprising a divalent metal ion comprises at least about 50 wt. %, at least about 60 wt. %, at least about 70 wt. %, at least about 80 wt. %, or at least about 90 wt. %, of the total metal silicate of the core shell silica particle.

48. The oral care composition according to claim 40, wherein the calcium-based abrasive system is present in an amount of from about 5 wt. % to about 50 wt. % of the oral care composition.

49. The oral care composition according to claim 40, wherein the amount of soluble divalent metal ion in the oral care composition is less than about 500 ppm, about 400 ppm, about 300 ppm, or about 250 ppm.

50. The oral care composition according to claim 40, wherein the oral care composition comprises a total zinc level of from about 0.1 wt. % to about 1 wt. %, of the oral care composition.

51. The oral care composition according to claim 40, wherein the amount of added water does not exceed 20 wt. %, of the oral care composition.

52. The oral care composition according to claim 40, further comprising an unbound metal silicate comprising a divalent metal ion.

53. An oral care composition comprising:
an orally acceptable carrier;
from about 5 wt. % to about 50 wt. % of a calcium-based abrasive system; and
a core shell silica particle comprising:
a metal silicate; and
a silica particle comprising a core having a surface;
wherein the metal silicate is chemically bound to a surface of the silica core;
wherein the metal silicate comprises a silicate of a Zn²⁺; and
wherein the total zinc level is from about 0.1 wt. % to about 1 wt. %, of the oral care composition.

54. The oral care composition according to claim 53, wherein the silica is selected from the group consisting of a precipitated silica, a fumed silica and a fused silica; and wherein the precipitated silica, fumed silica or fused silica has a first diameter.

55. The oral care composition according to claim 54, wherein the core shell silica particle has a second diameter.

56. The oral care composition according to claim 55, wherein the first diameter is greater than the second diameter.

57. The oral care composition according to claim 53, wherein the metal silicate layer has an uneven surface.

58. The oral care composition according to claim 40, wherein the oral care composition is in a form selected from: a paste; a gel; a prophy; a dissolvable strip; a tablet; a tape; floss; and a mouthwash or mouth rinse.

59. A method of treating, preventing, or ameliorating a symptom associated with, a disease, disorder or condition of the oral cavity, comprising:
administering a composition according to claim 40, to an oral surface of a mammal in need thereof;
wherein the disease, disorder or condition of the oral cavity is selected from: erosion; caries; inflammation; excessive tarter; gingivitis; periodontitis; dentin hypersensitivity; discolored teeth; xerostomia; bleeding gums; plaque overgrowth; and malodor.
